(19) United States
(12) Patent Application Publication
(10) Pub. No.: US 2007/0207105 A1
(43) Pub. Date: Sep. 6, 2007

(54) Anti-microbial compositions

(75) Inventor: Daniel Winn, Kingston, NJ (US)

Correspondence Address: FLASTER/GREENBERG P.C.;8 PENN CENTER, 1628 JOHN F. KENNEDY BLVD., 15TH FLOOR, PHILADELPHIA, PA, 19103, US

(73) Assignee: Inolex Investment Corporation, Wilmington, DE (US)

(21) Appl. No.: 11/716,261

(22) Filed: Mar. 9, 2007

Related U.S. Application Data
(63) Continuation of application No. PCT/US07/04325, filed on Feb. 21, 2007.
(60) Provisional application No. 60/775,605, filed on Feb. 21, 2006.

(51) Int. Cl. A61K 31/45; A61K 31/22; A61K 31/425; A61K 8/18

(52) U.S. Cl. 424/70.1; 514/372; 514/546; 514/723

(57) ABSTRACT

Cosmetic and/or toiletry formulations having novel preservative blends including isothiazolinone and vicinal diols which are liquid at room temperature. The formulations provide antimicrobial properties including unexpectedly enhanced antifungal properties.

### BACKGROUND OF THE INVENTION

The field of the invention relates to providing antimicrobial activity to compositions, particularly to cosmetic and toiletry products.

Cosmetic and toiletry products such as creams, lotions, pastes, liquids, aerosols, shampoos, gels, wipes, bats, sticks, powders and granules, are known in the art to be susceptible to microbial infestation. The raw materials, packaging, and manufacturing environment for cosmetics are often not sufficiently sterile, such that small amounts of microbiological contaminants can enter into final products. Shipment and storage of packaged cosmetic and/or toiletry products in some cases are performed under uncontrolled conditions. Often, a cosmetic and/or toiletry product may be exposed to higher temperatures than recommended which can also accelerate the growth rate of microbes unless a suitably effective antimicrobial component and/or components are incorporated into the formulation. Once cosmetic packages are opened, they are subject to further contamination from repeated consumer use. A consumer may notice microbial infestation by the discoloration and/or unpleasant odor of a cosmetic product, or they might see macroscopic quantities of microorganisms such as mold on the product. Microbial growth can also cause the degradation of chemical compounds in the cosmetic formulation, which can lead to instability of a cosmetic emulsion. A cosmetic or toiletry product that has been contaminated by microbiological organisms can also lead to user infections once it is applied to the skin, scalp and/or mucous membranes of a human.

It is therefore of extreme importance in the art for cosmetic manufacturers and marketers to be able to offer products that resist microbial growth and provide for a stable and safe product with a long shelf-life. Typically, cosmetic manufacturers add small amounts of one or more preservative compounds to the cosmetic formulation to prevent microbial growth. The preferred preservatives are water-soluble, since typically it is the water phase of a cosmetic product that is most susceptible to microbial growth. The preferred preservatives are those that, at low use levels, are effective in reducing the cost of the formulation and in preventing excessive irritation associated with many preservative compounds. The preferred preservatives are those that do not adversely affect the aesthetic properties of the cosmetic formulation such as the odor and color. Furthermore, it is also desirable that the preservative does not affect the performance attributes of the product.

Finally, such preservatives must follow the guidelines established by country laws and regulations. These regulations limit the type and use-level of preservatives. In some countries, certain preservatives are permitted only for rinse-off products (such as shower gels) but not for leave-on products (such as skin creams). Therefore, preferred preservatives would be those that are not forbidden in any country, and which are not restricted to only certain product forms.

Preservatives used in cosmetics and toiletries must also meet consumer preferences. In recent years cosmetic preservatives have been a frequent target of academics and activist groups who question their toxicological safety. The resulting media reports have suggested that certain cosmetic preservatives are dangerous. As a result, cosmetic manufacturers prefer to use preservatives not tainted by negative publicity and that will not adversely affect their products marketability.

Preservatives used in cosmetic and toiletry products must enable the products to successfully pass microbiological testing protocols, known as challenge tests, established by government regulations and trade organizations. Challenge tests are performed by adding known quantities of microorganism to a cosmetic product and measuring the increase or decrease in microorganism quantity over time. The organisms include gram-positive bacteria, gram-negative bacteria, yeast and mold. The Cosmetics, Toiletries, and Fragrance Association (CTFA) has defined a challenge test that is widely accepted as the standard in the industry. The test requires that the quantity of bacteria be reduced by 99% in seven days, and that the quantity of yeast and fungi (mold) be reduced by 90% in seven days. In order to pass a challenge test, the cosmetic product must contain the appropriate amounts and types of preservative compounds that will enable antimicrobial efficacy against a broad spectrum of microorganisms in short period of time.

In recent years cosmetic manufacturers have been severely limited in their choice of preservative agents. A class of biocides that have been highly effective in cosmetics are formaldehyde donors, such as imidazolidinyl urea, diazolidinyl urea, and DMDM hydantoin. However, many such compounds are considered to be skin irritants and the use of formaldehyde donors is severely restricted by regulations in the EU and Japan.

Another class of cosmetic preservatives are the isothiazolinones, such as Kathon® CG, available commercially from Rohm & Haas, Philadelphia, Pa., which contains a chloro-substituted isothiazolinone (methylchloroisothiazolinone). This chloro-substituted isothiazolinone has demonstrated irritation potential and it is prohibited from use in leave-on products in some countries.

Another class of cosmetic preservatives is chlorinated aromatic compounds, such as chlorphenesin. They are not broadly used in cosmetics because they exhibit a very strong and unpleasant odor. Also chlorinated compounds in general are used in herbicides and pesticides, and many are known human toxins, and thus chlorinated compounds may have a negative consumer perception.

Another class of cosmetic preservatives is para-hydroxybenzoic acids, known as parabens. Preservative blends containing parabens, such as Germall® and Liquapar®, available commercially from International Specialty Products, and Phenonip®, available commercially from Clariant, are the most widely used preservative systems and have been used safely and effectively for over 20 years. However, research reports such as the recent Journal of Applied Toxicology [2004, 24, 5] have suggested that parabens are possible human carcinogens. The media has suggested that cosmetics containing parabens are dangerous. Consumer groups, such as Breast Cancer Action, have lobbied cosmetic and toiletry companies to remove parabens from their products. As a result parabens are now defacto banned from many segments of the cosmetics and toiletry industry.

In U.S. Published Patent Application US-2005-0228032-A1, International Specialty Products describes a paraben-free, broad-spectrum preservative blend that includes glycols, phenoxyethanol and organic acids. In U.S. Pat. No. 6,447,793 B2, Engelhard Corporation describes paraben-free, broad-spectrum preservation systems that include phenoxyethanol, chlorphenesin and organic acids. Such blends fail to meet all of the industry-desired needs, because organic acids are only effective as biocides in cosmetic and toiletry products that have an acidic pH. Cosmetic and toiletry products having an acidic pH are limited to certain leave-on formulations, such as creams and lotions. Organic acids are completely ineffective biocides at neutral pH, and thus these preservative blends are not suitable for pH-neutral cosmetic and toiletry products, such as body washes and shampoos.

U.S. Patent Publication No. 2006-0106024-A1 discloses a microbicidal composition useful for inhibiting the growth of microorganisms or higher forms of aquatic life which includes 1,2-benzisothiazolin-3-one or 2-methyl-4-isothiazolin-3-one and a microbicide, wherein the microbicide is chosen from the following group: benzalkonium chloride, benzethonium chloride, benzyl alcohol, caprylyl glycol, chlorphenesin, 2,2′-dithiobis(N-methylbenzamide), diazolidinyl urea, ethylenediamine tetraacetic acid, ethylparaben, imidazolidinyl urea, methylparaben, phenoxyethanol, linoleamidopropyl PG-dimonium chloride phosphate, cocamidopropyl PG-dimonium chloride phosphate, propylparaben, cis-1-(3-chloroallyl)-3,5,7-triaza-1-azoniaadamantane chloride, dehydroacetic acid or its salts, benzoic acid or its salts, sodium hydroxymethylglycinate and zinc pyrithione. Of this group, caprylyl glycol is a vicinal diol which is typically commercially available as a solid at room temperature.

Therefore, a need in the art remains for preservative compositions that are free of parabens, formaldehyde donors and chlorinated compounds, that are globally approved for use in cosmetics in leave-on and rinse-off products, and that have efficacy against a broad spectrum of microorganisms at various levels of pH.

### BRIEF SUMMARY OF THE INVENTION

The invention includes a method of producing an antimicrobial composition, comprising providing a blend of a vicinal diol and an isothiazolinone compound to the composition.

Also within the invention is a method of producing an antimicrobial composition, comprising providing a blend of a vicinal diol, an isothiazolinone, and a non-vicinal glycol as a solubilizing agent.

A method for preserving a cosmetic and toiletry formulation is also disclosed, wherein the formulation is free of parabens, and the method comprises incorporating into the formulation a blend of a vicinal diol and an isothiazolinone.

A cosmetic and/or toiletry formulation free of parabens is also within the invention and comprises a preservative blend of an isothiazolinone and a vicinal diol.

### DETAILED DESCRIPTION OF THE INVENTION

The invention relates to anti-microbial compositions that include both an isothiazolinone biocide and a vicinal diol. The compositions have broad-spectrum antimicrobial activity necessary for the preservation of cosmetic and toiletry products. In particular, the invention includes compositions including combinations of the vicinal diols that are liquid at room temperature, such as 1,2-hexanediol or ethylhexylglycerin, in combination with the isothiazolinone, methylisothiazolinone. The combination of the isothiazolinone and the liquid vicinal diol provide a synergistically intensified antimicrobial effect.

In one preferred embodiment, 1,2-hexanediol is blended with methylisothiazolinone, and further blended with another vicinal diol such as ethylhexylglycerin, such that the entire blend is liquid and therefore easy to blend into a cosmetic emulsion. Additional solvents (which may also be vicinal diols in some cases) such as propylene glycol, butylene glycol, pentylene glycol, and hexylene glycol may be added to enhance water solubility.

“Vicinal diols,” as used herein, are materials that have hydroxyl groups which are bonded to atoms in the molecule which are next to each other, i.e. wherein two atoms each bearing a hydroxyl group are bonded to each other. Examples of vicinal diol compounds suitable for use in the invention, include but are not limited to, ethylene glycol and propylene glycol. Such materials are known for use as humectants and solvents in cosmetics and toiletry products. They are also known to have some modest antimicrobial activity. The most preferred vicinal diols for use in the compositions described herein when used in cosmetics applications are those linear vicinal diols that are liquid at room temperature and that are at least partially soluble in water, making them easy to formulate into cosmetics formulations. Such diols include 1,2-pentanediol and 1,2-hexanediol and other similar diols which are liquid at room temperature. Other vicinal diols useful in the compositions described herein include molecules derived from glycerin, such as, but not limited to, 1,2,3-propanetriol. Glycerin can be reacted with other molecules at its 1 or 3 position, leaving two vicinal hydroxyl groups. For example, glyceryl monoethers, such as ethylhexylglycerin [3-(2-ethylhexyloxy)propane-1,2-diol], available commercially as Sensiva®SC50 from Schulke & Mayr, are useful liquid vicinal diols having antimicrobial properties. Glyceryl monoesters which are liquid at room temperature may also be useful antimicrobial vicinal diols.

Isothiazolinones are a class of sulfur-containing biocides. Common isothizolinones derivatives include benzisothiazolinone (available commercially as Nipacide® BIT from Clariant and as Preventol® BIT from Lanxess) and n-octylisothiazolinone, which are industrial biocides not approved for use in cosmetics. One chlorinated derivative thereof, methylchloroisothiazolinone (Kathon® CG, available commercially from Rohm & Haas, Philadelphia, Pa.) is a cosmetic preservative, but it is not approved for all leave-on products in all countries. The isothiazolinone derivative for use in the preferred compositions described herein is methylisothiazolinone, because it is globally approved in leave-on and rinse-off cosmetic products. It is available commercially as Neolone® 950, from Rohm and Haas, Philadelphia, Pa. Neolone®950 product is a 9.5% solution of methylisothiazolinone in water. “Neolone” as used further herein below will refer to Neolone® 950, unless otherwise indicated.

Methylisothiazolinone is considered to have adequate effectiveness against bacteria and yeast, yet to have weak effectiveness against fungi. The manufacturer of Neolone®, Rohm and Haas, does not describe antifungal activity in its literature. In fact, the literature suggests using Neolone® with other preservatives, such as parabens, in cosmetics formulations in order to provide necessary antifungal activity. In the book, D. Steinberg, Preservatives for Cosmetics, 2nd ed, (2006) at pg. 28, the author states that methylisothiazolinone “ . . . is weakest against fungi. It should be used with antifungal preservatives.” Therefore, a cosmetic product with methylisothiazolinone as the only preservative is not expected pass the CTFA challenge test for fungi. A cosmetic product containing methylisothiazolinone in combination with other non-antifungal preservatives is also not expected to pass a CTFA challenge test.

Vicinal diols are similarly effective against bacteria and yeast but weak against fungi. In D. Steinberg, noted above at 102, the author comments regarding vicinal diols that “[t]he weakest activity on all of these is fungi.” In the article, D. Smith et al., “The Self-Preserving Challenge,” Cosmetic & Toiletries, No 1, 115, No. 5 (May 2000), vicinal diols are described as having activity against bacteria, but to be “[l]imited against Aspergillus.” Since Aspergillus Niger is one of the microorganisms used in the CTFA challenge test, cosmetics with vicinal diols as described herein as the only preservative would not be expected to pass the CTFA challenge test.

Surprisingly, despite the weak levels of activity in each of these compounds alone, the compositions described herein which include combinations of these two agents having inadequate anti-fungal activity in a cosmetic formulation are sufficient for cosmetic products to pass a CTFA challenge test. This combination of two non-antifungal preservatives showed particularly unexpected levels of effectiveness against fungi. Based on the prior belief in the art, it would be expected that if methylisothiazolinone were combined with another material expected to be weak against fungi, such as the vicinal diols described herein, that the combination also would not have adequate activity against fungi, in particular against Aspergillus Niger. However, unexpectedly, the present compositions provide the opposite effect.

Another surprising result achieved by compositions according to the invention is that antifungal activity levels were achievable at use levels below those known and expected in the art to have activity. In the book, D. Steinberg, Preservatives for Cosmetics, 2nd ed, (2006), pg. 102, it states that 1,2-hexanediol should be used at levels “>4%” in cosmetic formulations. However, compositions according to the present invention can incorporate vicinal diols such as 1,2-hexanediol at levels of only about 1% by weight, when used in combination with methylisothiazolinone, and provides properties that are sufficient to pass a CTFA challenge test.

It is preferred that in the present invention, the preferred cosmetic and/or toiletry compositions include from about 0.01 to about 10 percent by weight (on a wet basis), and more preferably from about 0.1 to about 5 weight percent, of the blend including at least one isothiazolinone and at least one vicinal diol, and any optional solubilizing agent as noted herein. While only one isothiazolinone is necessary, such materials as described above may be used alone or in combinations with each other and with one or more vicinal diols. The amount of the isothiazolinone and the vicinal diol components in the blend should be selected so as to preferably provide a ratio of isothiazolinone(s) to vicinal diol(s) in the blend which is to be provided to the composition of from about 99.999:0.001 to about 0.001:99.999 and more preferably from about 10.00:0.01 to about 0.01: 10.00, and most preferably from about 10.0:0.1 to about 0.1:1.0.

In one preferred embodiment, an additional vicinal diol is also incorporated into a blend of the vicinal diol and the isothiazolinone to make a three-component blend that is then incorporated into a cosmetic or toiletry formulation. In such an embodiment, the additional vicinal diol is included in the blend in an amount of about 1 percent by weight to about 70 percent by weight of the blend, based on the total weight of the blend. One preferred embodiment of a three-component preservative blend includes a combination of components in the blend of about 0.01 to about 50 by weight of an isothiazolinone (and a suitable amount of water if the isothiazolinone is used in an water-based solution), about 0.01 to about 98 by weight of a first vicinal diol and about 1 to about 70 percent by weight of a second vicinal diol. One preferred exemplary three-component blend includes from about 0.01 to about 20 percent by weight, more preferably about 0.01 to about 10 percent of methylthiazolinone (and an approximately an equal amount of water (preferably 40:60 to 60:40) if used in a water-based solution), about 10 to about 40 percent by weight, more preferably about 30 to about 40 percent of 1,2-hexanediol, and about 40 to about 90 percent by weight, preferably about 50 to about 70 percent by weight of ethylhexylglycerin.

Formulations prepared for cosmetic and/or toiletry compositions may include any other colorants, fragrances or other additives typically used and/or to be developed in the art for use in cosmetic and/or toiletry compositions, which additives will vary depending upon the formulation in which the preferred compositions are used, i.e., whether the formulations are used in skin treatments such as moisturizing compositions, skin toners, skin cleansers, night creams, skin creams, shaving creams, skin care lotions, or other cosmetic preparations; make-up, such as foundation, liquid and powder-based make-up, mascara, lipstick, blush, gloss, eye-liner and the like; or other toiletries such as sunscreens, lip balm, fragrances, massage oil, shampoos, conditioners, conditioning shampoos, hair styling gels, hair reparatives, hair tonics, hair fixatives, hair mousses, bath and shower gels, liquid soaps, moisturizing sprays, makeup, pressed powder formulations, bath additives, foaming soaps and body washes, sanitizing wipes, hand sanitizers, towelettes and wipes and others. It should be understood, based on this disclosure that a wide variety of cosmetic and toiletry formulations could benefit from the properties of the compositions of the present invention.

The formulations, if liquid based (such as gels, hydrogels, lotions, shampoos and the like) will also include water. The formulations and compositions may include other additives as well, such as without limitation, at least one humectant, at least one emulsifier and/or thickener, chelating agent(s), gelling agent(s), amino acid(s), emollient(s), various solvents, free radicals and initiators, sunscreen UVA and/or UVB blocking agents, antioxidants, other preservatives, waxes, polymers and copolymers, inorganic and organic pigments and/or one more fragrances, coloring agent(s), herbs and other additives commonly used in such formulations.

The compositions herein may be lotion-based, oil-in-water emulsions, water-in-oil emulsions, water-in-silicone emulsions, silicon-in-water emulsions, gels, solids, liquids, cream based, oil based, aqueous/alcoholic or glycolic solution based, a microemulsion or a liposome-based formulation.

In water-based formulations, other than solids and thicker gels, etc., it is preferred that from about 20% by weight to about 95% by weight (on a wet basis) of water is incorporated therein. The various additives aside from the water and preferred combination of preservatives noted herein including vicinal diols with isothiazolinones, would make up the remaining portion of various cosmetic and toiletry formulations based on the compositions described herein. Preferably each additive is present in an amount of up to about 75 percent by weight (on a wet basis) of the entire formulation, and more preferably up to about 40 percent by weight, with a collective amount of such additives of preferably no greater than about 50 percent by weight.

### EXAMPLE 1

The following skin care emulsion formulations were developed and then challenge tested. Table 1 describes two skin care formulations that are identical with the exception of their preservative system: Comparative Product A contains no preservative, while Inventive Product B contains 0.5% of a preservative blend. The preservative blend in Inventive Product B is a combination of 36% of 1,2-hexanediol, 4% of a 50% methylisothiazolinone solution in water, and 60% of ethylhexylgycerin. The formulations are shown below in Table 1.

TABLE 1

| | Comparative | Inventive |
|---|---|---|
| | Formulation A | Formulation B |
| | (% w/w) | (% w/w) |
| Deionized Water | Q.S. | Q.S. |
| Xanthan Gum | 0.40 | 0.40 |
| Glycerin 96% | 1.50 | 1.50 |
| Butylene Glycol | 1.00 | 1.00 |
| Tetrasodium EDTA | 0.10 | 0.10 |
| Preservative Blend (36% 1,2- | — | 0.50 |
| hexanediol, 2% | | |
| methylisothiazolinone and 2% | | |
| water, 60% | | |
| ethylhexylglycerin) | | |
| Octinoxate | 7.50 | 7.50 |
| Oxybenzone | 5.25 | 5.25 |
| Octisalate | 5.00 | 5.00 |
| Avobenzone | 2.00 | 2.00 |
| Homosalate | 13.00 | 13.00 |
| Glyceryl Stearate and PEG- | 2.50 | 2.50 |
| 100 Stearate | | |
| Neopentyl Glycol | 2.25 | 2.25 |
| Diheptanoate | | |
| Adipic Acid/Diethylene | 3.00 | 3.00 |
| Glycol/Glycerin | | |
| Crosspolymer | | |
| Hydroxyethylacrylate/Sodium | 3.50 | 3.50 |
| Acryloyldimethyltaurate | | |
| Copolymer and Squalane and | | |
| Polysorbate 60 | | |
| Silica | 2.00 | 2.00 |
| Total | 100.00 | 100.00 |

A challenge test complying with USP and CTFA methodologies was performed. The results are in Table 2. The table indicates the log value of the number of viable organisms measured after the expired time interval. The term TNTC is the acronym for “Too Numerous To Count” and indicates that the number of viable organisms has increased as compared to the initial inoculum. Product A, containing no preservative, fails to meet the CTFA acceptance criteria of a 99% reduction in bacteria and 90% reduction in yeast and fungi within 7 days. Product B, containing a preservative blend of vicinal diol and methylisothiazolinone according to the invention, meets the CTFA acceptance criteria. In particular it meets the acceptance criteria of greater than 90% reduction in fungus (mold) in 7 days, with no increase in mold thereafter up to 28 days.

TABLE 2

| | S. Aureus | | E. Coli | | P. Aeruginosa | | C. Albicans | | A. Niger | |
|---|---|---|---|---|---|---|---|---|---|---|
| | A | B | A | B | A | B | A | B | A | B |
| Inoculum | 5.71 | 5.58 | 5.95 | 5.93 | 5.77 | 5.92 | 5.77 | 5.2 | 5.3 | 5.00 |
| Day 7 | TNTC | 2.27 | TNTC | <1.0 | TNTC | <1.0 | TNTC | <1.0 | TNTC | 2.47 |
| Day 14 | 4.41 | <1.0 | 5.17 | <1.0 | 2.53 | <1.0 | 5.03 | <1.0 | 4.9 | 1.74 |
| Day 21 | Failed | <1.0 | Failed | <1.0 | Failed | <1.0 | Failed | <1.0 | Failed | <1.0 |
| Day 28 | Failed | <1.0 | Failed | <1.0 | Failed | <1.0 | Failed | <1.0 | Failed | <1.0 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method of producing an antimicrobial composition, comprising
providing a blend of a vicinal diol which is a liquid at room temperature and an isothiazolinone compound to the composition.

2. The method according to claim 1, wherein the vicinal diol is a 1,2-alkanediol.

3. The method according to claim 2, wherein the 1,2-alkanediol is selected from the group consisting of 1,2-pentanediol and 1,2-hexanediol.

4. The method according to claim 1, wherein the vicinal diol is a glyceryl monoester or glyceryl monoether.

5. The method according to claim 4, wherein the vicinal diol is the glyceryl monoether and is ethylhexylglycerin.

6. The method according to claim 1, wherein the isothiazolinone is a methylisothiazolinone.

7. The method according to claim 6, wherein the methylisothiazolinone has a structure, 2-Methyl-3(2H)isothiazolinone.

8. The method according to claim 1, wherein the vicinal diol comprises about 0.001% by weight to about 99.999% by weight of the blend.

9. The method according to claim 1, wherein the isothiazolinone comprises about 0.001% to about 99.999% by weight of the blend.

10. A method of producing an antimicrobial composition, comprising providing a blend of a vicinal diol which is a liquid at room temperature, an isothiazolinone, and a second vicinal diol.

11. The method according to claim 10, wherein the glycol solubilizing agent comprises about 1% to about 70% by weight of the blend.

12. A method for preserving a cosmetic and toiletry formulation, wherein the formulation is free of parabens, comprising incorporating into the formulation a blend of a vicinal diol which is liquid at room temperature and an isothiazolinone.

13. The method according to claim 12, further comprising incorporating a second vicinal diol into the blend.

14. The method according to claim 12, wherein the blend comprises about 0.01 to about 5% by weight of the cosmetic or toiletry formulation on a wet basis.

15. A cosmetic and/or toiletry formulation free of parabens, comprising a preservative blend of an isothiazolinone and a vicinal diol, wherein the vicinal diol is liquid at room temperature.

16. The cosmetic and/or toiletry formulation according to claim 15, wherein the blend further comprises a second vicinal diol.

17. A blend for use in a cosmetic and/or toiletry formulation, comprising:
(a) isothiazolinone;
(b) a first vicinal diol; and
(c) a second vicinal diol.

18. The blend according to claim 17, wherein the isothiazolinone is methylisothiazolinone, the first vicinal diol is 1,2-hexane diol and the second vicinal diol is ethylhexylglycerin.

19. The blend according to claim 18, wherein the blend comprises about 0.01 to about 20 percent by weight of the methylisothiazolinone, about 0.01 to about 20 percent by weight water, about 10 to about 40 percent by weight of the 1,2-hexanediol, and about 40 to about 90 percent by weight of the ethylhexylglycerin.

20. The blend according to claim 17, wherein the isothiazolinone comprises about 0.01 to about 50 by weight of the blend, the first vicinal diol comprises about 0.01 to about 98 percent by weight of the blend and the second vicinal diol comprises about 1 to about 70 percent by weight of the blend.
